(19) United States
(12) Patent Application Publication
(10) Pub. No.: US 2011/0311488 A1
(43) Pub. Date: Dec. 22, 2011

(54) MODIFIED BACTERIAL CELL

(75) Inventors: Stephen Diggle, Nottingham (GB); Shanika Crusz, Nottingham (GB); Stuart West, Oxford (GB); Ashleigh Griffin, Oxford (GB); Kendra Rumbaugh, Lubbock, TX (US)

(73) Assignees: The University of Nottingham, Nottingham (GB); Texas Tech University System Office of Technology Transfer and Intellectual Property, Lubbock, TX (US); University Court of the University of Edingburgh, Edinburgh (GB)

(21) Appl. No.: 13/120,062

(22) PCT Filed: Sep. 22, 2009
(86) PCT No.: PCT/GB2009/002238

§ 371 Date: Sep. 6, 2011

(30) Foreign Application Priority Data
Sep. 22, 2008 (GB) 0817240.5

(51) Int. Cl. A61K 35/74; A61P 31/04; A61P 29/00; A01P 1/00; A61P 1/04; A61P 31/06; C12N 1/21; A01N 63/00

(52) U.S. Cl. 424/93.2; 435/252.3

(57) ABSTRACT

We describe modified bacterial cells that are defective in gene expression and their use in the treatment of bacterial infections of animals and plants and the inhibition of bacterial biofilm formation.

The invention relates to modified bacterial cells that are defective in gene expression, for example defective in gene expression related to quorum sensing [QS], and their use in the treatment of bacterial infections of animals and plants and the inhibition of bacterial biofilm formation.

Antibiotics have provided a significant contribution to the control of bacterial infection since the identification of penicillin in the 1930s. However, current antibiotic treatments are now prejudiced by the emergence of drug-resistant bacteria. The extensive use of antibiotics in the treatment of human and animal disease has placed a selective pressure on bacteria resulting in the evolution of bacterial genes that confer resistance to one or more antibiotics resulting in multi-resistant bacterial species. The rapid transfer of mutated resistance genes by horizontal transfer of plasmids that encode resistance genes compounds the problem. This is a major social and medical problem. There is therefore a continued need to identify and develop new antibiotics and treatments that combat bacterial antibiotic resistance.

There are many examples of bacterial species that have developed antibiotic resistance. For example the genus Pseudomonas spp includes a large number of pathological species that infect humans, animals and plants. The Pseudomonads are naturally resistant to penicillin and related antibiotics. P. aeruginosa is an opportunistic human pathogen that has recently become significant in a clinical context and inherently has a low susceptibility to antibiotics and can easily develop multi-resistance to commonly used antibiotics. P. aeruginosa chronically persists on medical devices and during infections such as those in the cystic fibrosis lung [CF] and in industrial settings by forming multicellular biofilms. During the formation of biofilms, cells abandon the isolation of the planktonic mode of growth and group together to form organised ‘slime-cities’. These complicated structures often contain channels for the import of nutrients and the disposal of waste products and they may even contain specialist cells, which appear to have specific roles within the biofilm. Medically, biofilms are of huge importance as they are capable of forming in the lungs of chronically ill patients such as those with CF or Congestive Obstructive Pulmonary Disease [COPD] and in chronic wounds [e.g. diabetic ulcers]. This is especially problematic as they are often resistant to desiccation and treatment with biocides and antibiotics.

As mentioned above the Pseudomonads are also significant plant pests causing damage to a large number of commercially relevant crops. P. syringae strains exist that have a high level of plant species specificity.

A further example of a pathogenic bacterium which has developed resistance to antibiotics is Staphylococcus spp. S. aureus is a bacterium whose normal habitat is the epithelial lining of the nose in about 20-40% of normal healthy people and is also commonly found on skin usually without causing harm. However, in certain circumstances, particularly when skin is damaged, this pathogen can cause infection. This is a particular problem in hospitals where patients may have surgical procedures and/or be taking immunosuppressive drugs. These patients are much more vulnerable to infection with S. aureus because of the treatment they have received. Resistant strains of S. aureus have arisen in recent years. Methicillin resistant strains are prevalent and many of these resistant strains are also resistant to several other antibiotics. S. aureus is therefore a major human pathogen capable of causing a wide range of diseases some of which are life threatening diseases including septicaemia, endocarditis, arthritis and toxic shock.

Additionally a non exhautive list of bacterial species that have developed antibiotic resistance includes: Enterococcus faecalis; Mycobacterium tubercuolsis; Streptococcus group B; Streptoccocus pneumoniae; Helicobacter pylori; Neisseria gonorrhoea; Streptococcus group A; Borrelia burgdorferi; Coccidiodes immitis; Histoplasma sapsulatum; Klebsiella edwardii; Neisseria meningitidis type B; Proteus mirabilis; Shigella flexneri; Escherichia coil; Haemophilus influenzae, Chalmydia trachomatis, Chlamydia pneumoniae, Chlamydia psittaci, Francisella tularensis, Pseudomonas aeruginos, Bacillus anthracis, Clostridium botulinum, Yersinia pestis, Burkholderia mallei or B pseudomallei.

Bacterial cells produce and secrete a number of factors that enhance bacterial growth, for example siderophores which are iron chelating proteins and other proteins that faciliate growth and virulence. Bacterial cells are able to communicate with one another within a community to co-ordinate the growth and physiology of the culture as a whole. This strategy is termed Quorum Sensing [QS] and allows bacteria to control gene expression in response to the level of diffusible signalling molecules called “autoinducers” that bind receptors presented by the bacterial cells within the community. Processes that are controlled by QS include virulence, bioluminescence, bioflim formation, swarming, sporulation, and plasmid transfer. QS is therefore of fundemental importance to the control of bacterial growth.

QS plays a key role in determining the damage that pathogenic bacteria inflict upon their hosts (virulence)¹⁻⁵. Work with animal models has shown that infections initiated with only QS mutants, that either do not produce or respond to autoinducer molecules, have significantly reduced virulence¹³. It has also been shown that QS mutants, especially those that that do not respond to autoinducer molecules (signal-blind), arise and spread during infections of humans¹⁰⁻¹². One possible explanation for this invasion is that the loss of QS is an adaptation to the host environment. An alternative possibility is that these mutants are social cheats that can outcompete wild type strains by exploiting their cooperative production of exoproducts^{2, 6, 7}. Cheats would increase in relative frequency because they benefit from the exo-products produced by others, while avoiding the cost of producing them. In this scenario, the ability of cheats to exploit others would have both a short and long term influence on the evolution of parasite virulence. In particular, a higher relatedness between the bacteria infecting a host (lower strain diversity) will favour higher levels of cooperation, that allow the host to be exploited more efficiently, and hence a higher virulence¹⁴. This contrasts with the standard prediction from evolutionary theory, in the opposite direction, that higher relatedness will lead to more prudent exploitation of the host, and hence lower virulence^{15, 16}.

The inhibition of QS by agents to control bacterial growth is known in the art. For example, WO2006/079015 describes compounds that modulate QS thereby affecting the virulence of bacteria and their sensitivity to antibiotics or the host's immune system; also see WO2006/078986 and WO2006/078904 for related compounds and WO2008/069374 which describes antagonists useful in the disruption of bacterial biofilms. W002/16623 discloses autoinducer inactivation proteins useful in the inactivation of N-acyl homoserine lactone autoinducers isolated from Bacillus thuringiensis. EP 1 795 205 describes a method to inhibit QS by exposure to one or more catalytic enzymes having activity with respect to QS autoinducers. WO2008/066631 describes bacterial mutants defective in QS sensing by introduction of mutations into the LuxR. The mutated strains show growth advantages useful in fermentation.

This disclosure relates to bacterial mutants, for example Pseudomonas aeruginosa mutants, to determine the consequences for virulence in infections. We show that in mixed infections, containing quorum sensing bacteria and cheats who do not respond to signal, virulence is reduced to that of an infection containing only cheats. We show that this is because cheats, that do not respond to signal, exploit the cooperative production of virulence factors by others, and hence increase in frequency. This reduces the overall spread and virulence of the bacterial infection. Our results explain the invasion of QS mutants in infections of humans¹⁰⁻¹² and suggest a novel therapy for treating bacterial infections.

According to an aspect of the invention there is provided a bacterial cell the genome of which is modified by addition, deletion or substitution of at least one nucleotide base in at least one site in the genome wherein said modification provides a cell defective in the expression of at least one gene for use in the inhibition of a bacterial infection.

In a preferred embodiment of the invention said use is as a medicament in the treatment of animal or human infection.

In an alternative preferred embodiment of the invention said bacterial infection is a pathological infection of a plant.

It will be apparent that means to effect said modification are well known in the art. For example the insertion of genetic material into an operon or regulatory sequence that controls expression of an operon may be undertaken by transposon integration. Additionally, or alternatively, the operon may be altered to provide for deletion of at least part of at least one gene located in the operon by homologous recombination with at least one suitably designed vector and/or the replacing of at least part of at least one gene located in an operon with homologous DNA carrying, for example, a translation termination codon thus preventing synthesis of a functional protein. Additionally or alternatively, the operon may be altered by base substitution and/or mutation by random or site-directed mutagenesis. In addition this disclosure includes naturally occuring bacterial isolates that have lost gene function which are known to exist in the art.

In a preferred embodiment of the invention said bacterial cell is Gram negative.

In an alternative preferred embodiment of the invention said bacterial cell is Gram positive.

In a preferred embodiment of the invention said bacterial cell is selected from the genus group consisting of: Enterococcus spp; Mycobacterium spp; Streptococcus group B spp; Streptoccocus spp; Helicobacter spp; Neisseria spp; Streptococcus group A spp; Borrelia spp; Coccidiodes spp; Histoplasma spp; Klebsiella spp; Proteus spp; Shigella spp; Escherichia spp; Haemophilus spp; Chalmydia spp; Francisella spp; Pseudomonas spp; Bacillus spp; Clostridium spp; Yersinia spp; Burkholderia spp; Pseudomonas spp; Staphylococcus spp; Listeria spp; Bradyrhizobium spp; Rickettsia spp; Bordetella spp; Campylobacter spp; Salmonella spp; Legionella spp; Vibrio spp; Xanthomonas spp; Agrobacterium spp; Rhizobium spp.

In a preferred embodiment of the invention said bacterial cell is from the genus Pseudomonas spp.

Preferably said bacterial cell is selected from the group consisting of: Pseudomonas aeruginosa; P. oryzihabitans, P. luteola, or P. putida.

Alternatively said bacterial cell is selected from the group consisting of: P. syringae, P. tolaasii, P. agarici, P. putida.

In an alternative preferred embodiment of the invention said bacterial cell of the genus Agrobacterium.

Preferably said bacterial cell is Agrobacterium tumefaciens.

In an alternative preferred embodiment of the invention said bacterial cell is from the genus Staphylococcus spp.

Preferably said bacterial cell is selected from the group consisting of: Staphylococcus aureus; S. epidermidis, S. hominis; S. haemolyticus; S. warneri; S. capitis; S. saccharolyticus; S. auricularis; S. simulans; S. saprophyticus; S. cohnii; S. xylosus; S. cohnii; S. warneri; S. hyicus; S. caprae; S. gallinarum; S. intermedius; or S. hominis.

In a further preferred embodiment of the invention said staphylococcal cell is S. aureus or S. epidermidis.

In a preferred embodiment of the invention said genome modification is to an operon comprising a gene that encodes a polypeptide that mediates QS.

Pseudomonas aeruginosa has two well characterised QS systems called Las and Rhl that control virulence factors [reviewed in Current Science 2006 vol 90, no 5, p 666-676]. Both LasI and RhlI encode autoinducer synthases that catalyse the formation of N-(3-oxododecanoyl)-homoserine [3O-C12-HSL] and N-(butanyol)-homoserine lactone [C4-HSL] respectively. When bacterial cultures reach high cell density LasR binds 3O-C12-HSL which co-operatively bind promoter elements up stream of genes that encode virulence related factors such as elastase [lasB], a protease [lasA] exootoxin A [toxA]. The second system, rhl, is controlled by the regulator RhlR. When activated by the autoinducer [C4-HSL] RhlR enhances the expression of rhamnolipid biosynthesis, elastase, pyocyanin and its autoinducer synthetase (rhlI). The las system also enhances expression of rhlR and rhlI. A third signal, the Pseudomonas quinolone signal (PQS), is also intricately involved with the las and rhl quorum-sensing systems. PQS also regulates the expression of LasB elastase and other virulence determinants and is governed by the las system and requires the presence of RhlR. Gram-positive bacteria [e.g. Staphylococcus] also utilize QS. For example, a number of Gram-positive organisms have been shown to employ small, modified oligopeptides as extracellular signalling molecules. These peptides activate gene expression by interacting with two-component histidine protein kinase signal transduction systems. For example, in Staphylococcus aureus, the expressions of a number of cell density-dependent virulence factors are regulated by the global regulatory locus agr (accessory gene regulator).

In a preferred embodiment of the invention said operon is the las operon.

In an alternative preferred embodiment of the invention said operon is the rhl operon.

In a preferred embodiment of the invention said gene is selected from the group consisting of: lasI and/or rhlI and/or lasR and/or rhlR

In a preferred embodiment of the invention said gene is lasR and/or lasI.

In an alternative preferred embodiment of the invention said gene is rhlR and/or rhlI.

In a preferred embodiment of the invention said gene comprises or consists of a nucleic acid sequence selected from the group consisting of:
i) a nucleic acid molecule comprising a nucleic acid sequence as represented in FIG. 5;
ii) a nucleic acid molecule comprising a nucleic acid sequence that hybridizes under stringent hybridization conditions to the nucleic acid molecule in i) above and encodes a polypeptide with the activity of a las autoinducer.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (Allows Sequences that Share at Least 80% Identity to Hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (Allows Sequences that Share at Least 50% Identity to Hybridize)
Hybridization: 6×SSC at RT to 55° C. for 16-20 hours
Wash at least twice: 2×-3×SSC at RT to 55° C. for 20-30 minutes each.

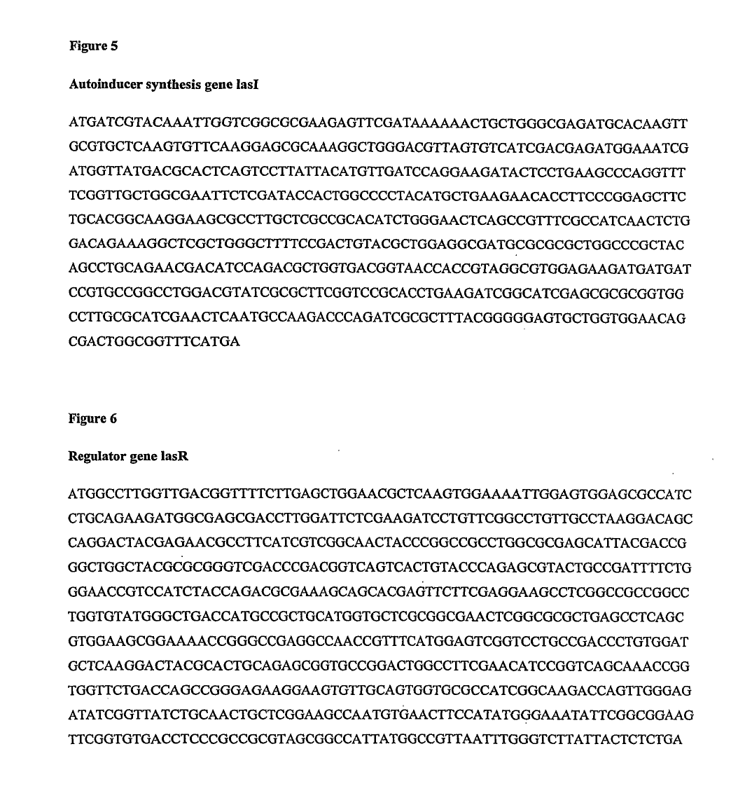

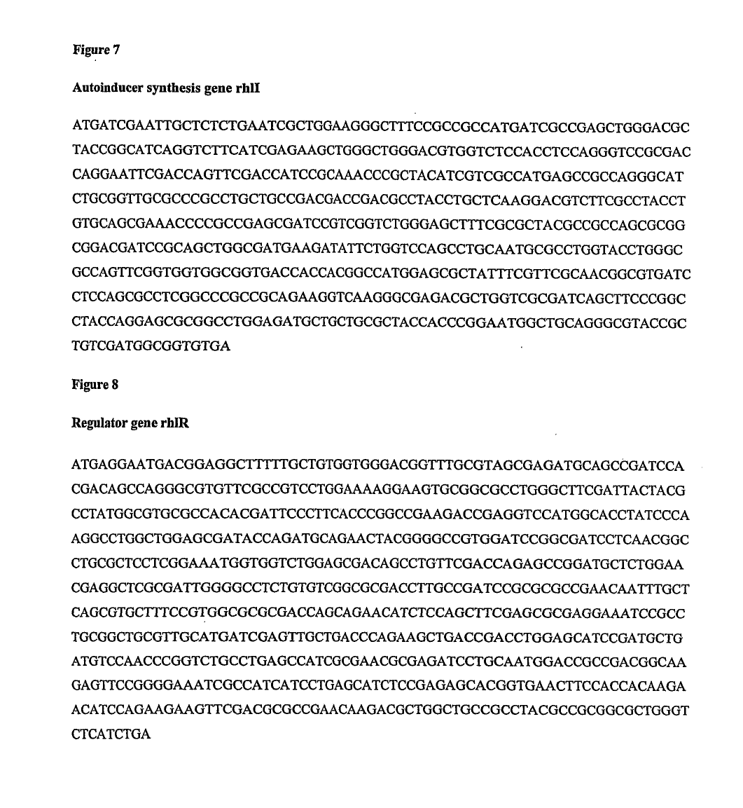

In an alternative preferred embodiment of the invention said gene comprises or consists of a nucleic acid sequence selected from the group consisting of:
i) a nucleic acid molecule comprising a nucleic acid sequence as represented in FIG. 6;
ii) a nucleic acid molecule comprising a nucleic acid sequence that hybridizes under stringent hybridization conditions to the nucleic acid molecule in i) above and encodes a polypeptide with the activity of a las regulator.

In a further preferred embodiment of the invention said gene comprises or consists of a nucleic acid sequence selected from the group consisting of:
a nucleic acid molecule comprising a nucleic acid sequence as represented in FIG. 7;
ii) a nucleic acid molecule comprising a nucleic acid sequence that hybridizes under stringent hybridization conditions to the nucleic acid molecule in i) above and encodes a polypeptide with the activity of a rhlI autoinducer.

In a preferred embodiment of the invention said gene comprises or consists of a nucleic acid sequence selected from the group consisting of:
i) a nucleic acid molecule comprising a nucleic acid sequence as represented in FIG. 8;
ii) a nucleic acid molecule comprising a nucleic acid sequence that hybridizes under stringent hybridization conditions to the nucleic acid molecule in i) above and encodes a polypeptide with the activity of a rhlI regulator.

In a preferred embodiment of the invention said genome modification is to an operon comprising a gene that encodes a polypeptide that encodes a siderdophore polypeptide.

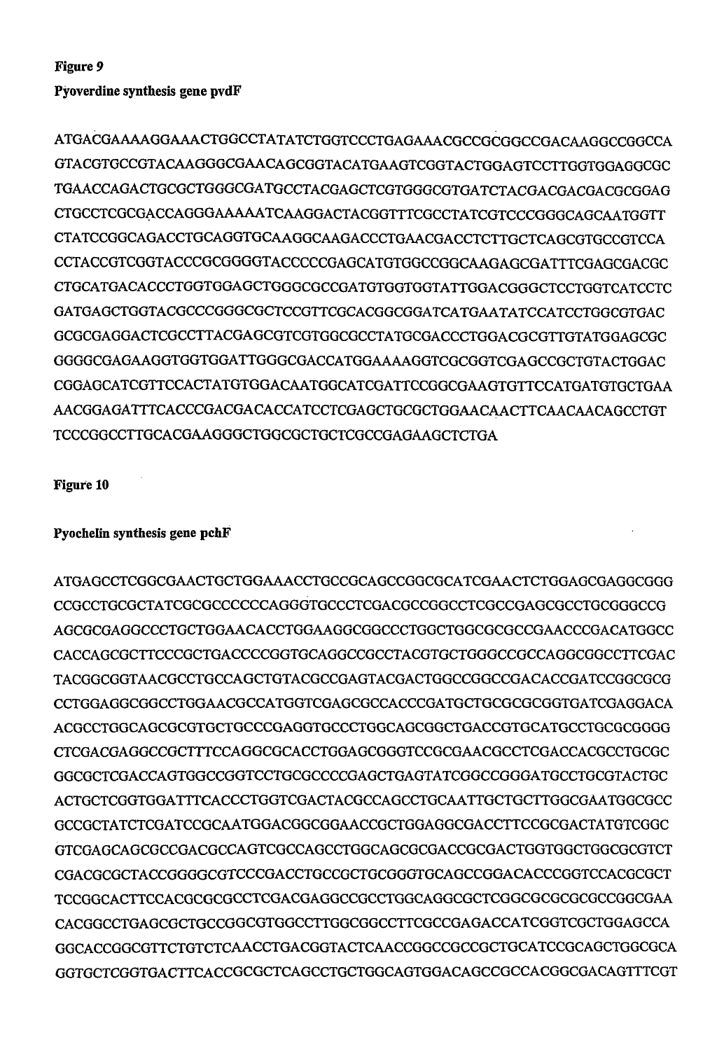

In a preferred embodiment of the invention said gene comprises or consists of a nucleic acid sequence selected from the group consisting of:
i) a nucleic acid molecule comprising a nucleic acid sequence as represented in FIG. 9;
ii) a nucleic acid molecule comprising a nucleic acid sequence that hybridizes under stringent hybridization conditions to the nucleic acid molecule in i) above and encodes a polypeptide with siderophore activity.

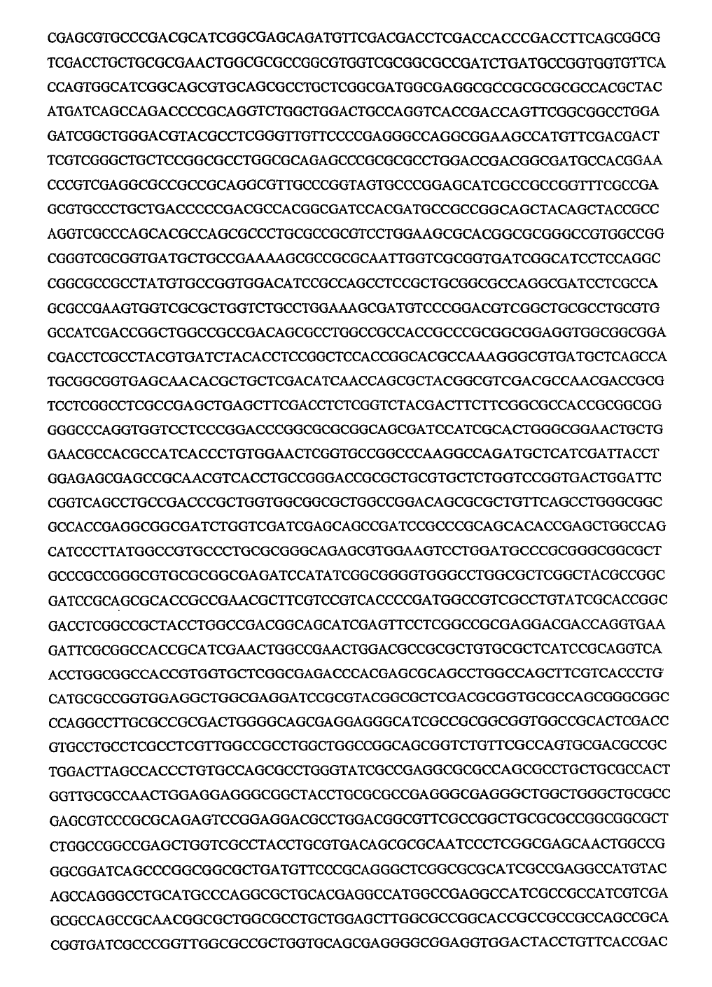

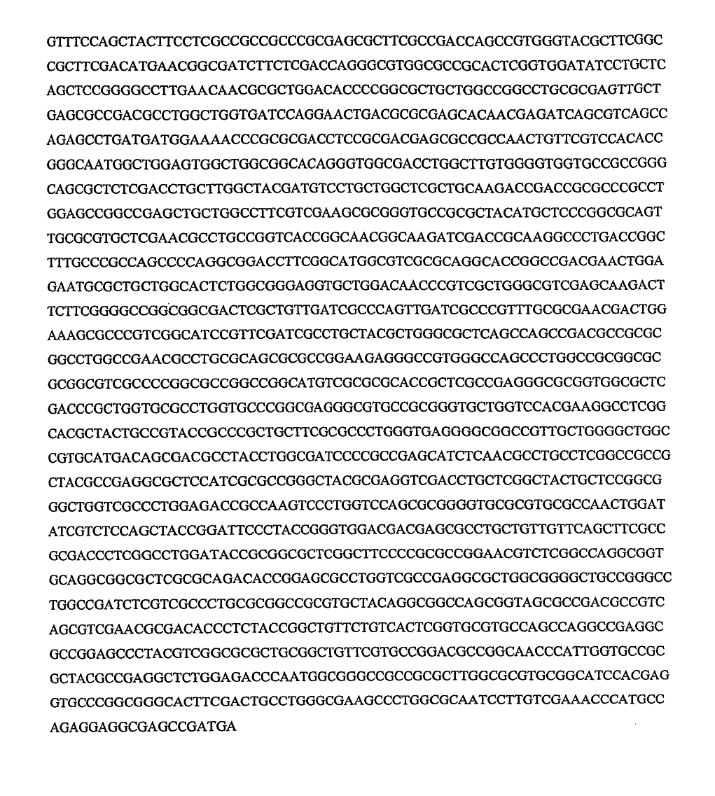

In a preferred embodiment of the invention said gene comprises or consists of a nucleic acid sequence selected from the group consisting of:
i) a nucleic acid molecule comprising a nucleic acid sequence as represented in FIG. 10;
ii) a nucleic acid molecule comprising a nucleic acid sequence that hybridizes under stringent hybridization conditions to the nucleic acid molecule in i) above and encodes a polypeptide with siderophore activity.

In a further preferred embodiment of the invention said modified bacterial cell is additionally modified by transformation with a nucleic acid molecule that encodes an agent which when expressed sensitizes said cell to an agent that inhibits the growth of said modified cell.

The invention includes modified bacterial cells that in addition are modified to prevent or inhibit the expression of genes important in the establishment of a bacterial infection are also modified to include nucleic acids that sensitize the modified bacterial cells to agents that control the growth of the modified bacterial cell. For example, the sacBR locus could provide a method for inhibiting the growth of a modified cell if engineered into the modified cells chromosome. The sacB gene encodes levanosucrase which catalyses the hydrolysis of sucrose. The action of this enzyme, in the presence of sucrose, is lethal to a large number of bacteria including P. aeruginosa (Kaniga et al., 1991, Gene 109 (1) p 137). Thus a bacterium containing this locus can only survive in the absence of sucrose in the environment. Addition of sucrose will kill any cells containing this locus.

According to a further aspect of the invention there is provided a therapeutic composition comprising a bacterial cell the genome of which is modified by addition, deletion or substitution of at least one nucleotide base in at least one site in the genome wherein said modification provides a bacterial cell defective in the expression of at least one gene and at least one therapeutic agent.

“Therapeutic agent” is to be understood to mean an antibiotic, anti-viral, anti-fungal or anti-cancer agent. Many bacterial infections are opportunistic and become resilient to treatment due in part to the immuno-suppressant state of the subject either due to the treatment administered [e.g. cancer as a consequence of chemotherapy and associated neutropenia and/or lymphopenia] or as a consequence of a disease that results in immune supression, for example AIDS. Therapeutic agent includes bacterial antibiotics, chemotherapeutic agents, biopharmaceuticals [e.g. cytokines], therapeutic monoclonal antibodies and therapeutic vaccines. Therapeutic vaccines will include carriers and adjuvants that enhance the immune response to the antigen contained in the vaccine.

In a preferred embodiment of the invention said therapeutic agent is an antibiotic.

In a preferred embodiment of the invention said antibiotic is tetracycline.

In an alternative preferred embodiment of the invention said antibiotic is gentamycin.

According to a further aspect of the invention there is provided a bacterial cell the genome of which is modified by addition, deletion or substitution of at least one nucleotide base in at least one site in the genome wherein said modification provides a bacterial cell defective in the expression of at least one gene and wherein said cell is combined with a delivery vehicle.

“Delivery vehicle” means a device to facilitate delivery of a modified bacterial cell according to the invention to a subject. For example, a prosthesis, implant, matrix, stent, gauze, bandage, plaster, biodegradable matrix, hydrogel. Hydrogels are amorphous gels or sheet dressings which are crosslinked and which typically consist of a polymer, a humectant and water in varying ratios. Hydrogels are known in the art and are commercially available. Examples of commercially available hydrogels are Tegagel™, Nu-Gel™ or FlexiGel™.

According to a further aspect of the invention there is provided the use of a bacterial cell the genome of which is modified by addition, deletion or substitution of at least one nucleotide base in at least one site in the genome wherein said modification provides a bacterial cell defective in the expression of at least one gene for the treatment of bacterial infection.

In a preferred embodiment of the invention said bacterial infection is selected from the group consisting of: septicaemia; tuberculosis; bacteria-associated food poisoning; blood infections; peritonitis; endocarditis; sepsis; bacterial meningitis; pneumonia; stomach ulcers; gonorrhoea; strep throat; streptococcal-associated toxic shock; necrotizing fasciitis; impetigo; histoplasmosis; Lyme disease; gastro-enteritis; dysentery; or shigellosis.

In a preferred embodiment of the invention there is provided antibiotic that is administered with said bacterial cell.

Preferably said antibiotic is administered simultaneously, (as an admixture), separately or sequentially to a subject.

In a preferred embodiment of the invention said bacterial infection is an opportunistic bacterial infection.

According to an aspect of the invention there is provided the use of a bacterial cell the genome of which is modified by addition, deletion or substitution of at least one nucleotide base in at least one site in the genome wherein said modification provides a bacterial cell defective in the expression of at least one gene in the treatment of wounds.

In a preferred embodiment of the invention said wound has a pre-existing bacterial infection.

In a preferred embodiment of the invention said wound is a burn or scald.

In an alternative preferred embodiment of the invention said wound is an ulcer, for example a diabetic ulcer.

According to a further aspect of the invention there is provided the use of a bacterial cell the genome of which is modified by addition, deletion or substitution of at least one nucleotide base in at least one site in the genome wherein said modification provides a bacterial cell defective in the expression of at least one gene for the inhibition of growth of plant bacterial pathogen.

In a preferred embodiment of the invention said bacterial cell is P. syringae.

According to a further aspect of the invention there is provided a method to control the growth of a plant bacterial pathogen comprising:
i) providing a bacterial cell the genome of which is modified by addition, deletion or substitution of at least one nucleotide base in at least one site in the genome wherein said modification provides a bacterial cell defective in the expression of at least one gene;
ii) contacting said cell with a plant; and optionally
iii) contacting said plant with a further agent that inhibits the growth of said plant pathogen.

In a preferred method of the invention said bacterial cell is P. syringae.

In an alternative preferred method of the invention said bacterial cell of the genus Agrobacterium; preferably Agrobacterium tumafaciens

In a further preferred method of the invention said plant is selected from the group consisting of: In a preferred embodiment of the invention said plant cell is selected from the group consisting of: In a preferred embodiment of the invention said plant is selected from the group consisting of: corn (Zea mays), canola (Brassica napus, Brassica rapa ssp.), flax (Linum usitatissimum), alfalfa (Medicago sativa), rice (Oryza sativa), rye (Secale cerale), sorghum (Sorghum bicolor, Sorghum vulgare), sunflower (Helianthus annus), wheat (Tritium aestivum), soybean (Glycine max), tobacco (Nicotiana tabacum), potato (Solanum tuberosum), peanuts (Arachis hypogaea), cotton (Gossypium hirsutum), sweet potato (lopmoea batatus), cassava (Manihot esculenta), coffee (Cofea spp.), coconut (Cocos nucifera), pineapple (Anana comosus), citris tree (Citrus spp.) cocoa (Theobroma cacao), tea (Camellia senensis), banana (Musa spp.), avacado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Prunus amygdalus), sugar beets (Beta vulgaris), sugar cane, oats, barley, vegetables and ornamentals.

Preferably, plants of the present invention are biomass crops (switchgrass, alfalfa, willow, poplar, eucalyptus, miscanthus, wheat, maize or barley.), other crop plants (for example, cereals and pulses, maize, wheat, potatoes, tapioca, rice, sorghum, millet, cassava, barley, pea), and other root, tuber or seed crops. Important seed crops are oil-seed rape, sugar beet, maize, sunflower, soybean, sorghum, and flax (linseed).

Horticultural plants to which the present invention may be applied may include lettuce, endive, and vegetable brassica including cabbage, broccoli, and cauliflower. The present invention may be applied in tobacco, cucurbits, carrot, strawberry, sunflower, tomato, pepper.

According to a further aspect of the invention there is provided the use of a bacterial cell the genome of which is modified by addition, deletion or substitution of at least one nucleotide base in at least one site in the genome wherein said modification provides a bacterial cell defective in the expression of at least one gene for the inhibition of biofilm formation.

According to a further aspect of the invention there is provided a method to inhibit the formation of a bacterial biofilm comprising:
i) providing a bacterial cell the genome of which is modified by addition, deletion or substitution of at least one nucleotide base in at least one site in the genome wherein said modification provides a bacterial cell defective in the expression of at least one gene;
ii) contacting said cell with a bacterial biofilm; and optionally
iii) adding an agent that disrupts said biofilm.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following table and figures:

Table 1 is a summary of clinical isolate signal molecule status with respect to quorum sensing signal molecules 3O-C12-HSL, C4-HSL, HHQ and PQS when grown as broth cultures;

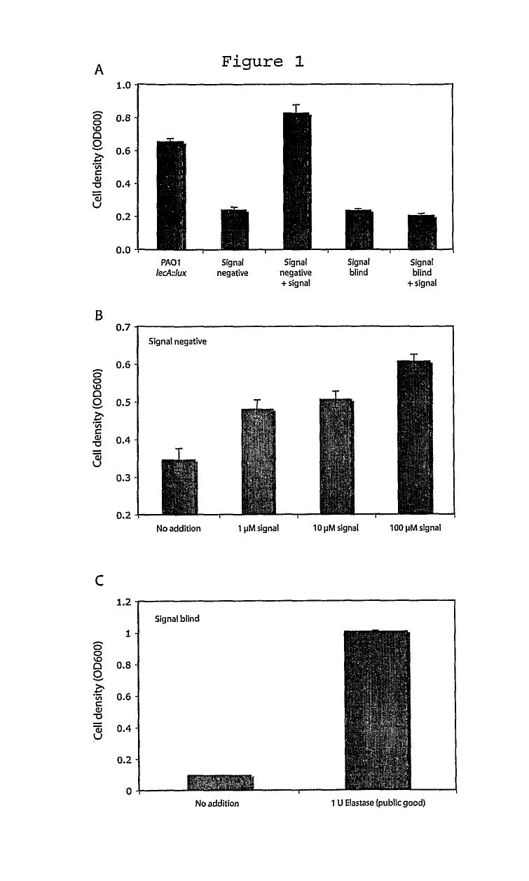

FIG. 1 illustrates the virulence of QS mutants. The survival rate for mice (burn model) infected with either a normal PA14 wild type or QS mutants, plotted against time (days post-burn/infection). Fifteen mice per treatment. The death rate does not vary significantly between the different mutants (X₃²=3.43, P>0.3), but is significantly lower in the mutants compared with the wild type (X₁²=6.54, P=0.01);

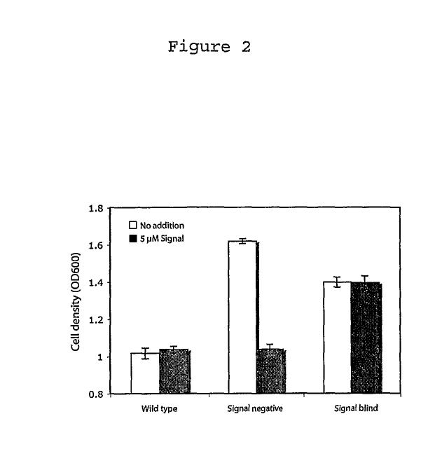

FIG. 2 illustrates the virulence of mixed infections. The survival curves for mice (burn model) infected with either the PA14 wild type, a QS mutant, or a 50:50 mixture of the two, plotted against time (days post-burn/infection). Shown are data for (a) the signal-blind (lasR) and (b) the signal-negative (lasI) mutants. Nine mice per treatment. In both cases, the survival rate of mice infected with the 50:50 mixture of wild type and mutant is significantly greater than that of the wild type (lasR: X₁²=13.91, P=0.0002; lasI: X₁²=6.24, P=0.02) and not significantly different from the mutant (lasR: X₁²=0.23, P>0.6; lasI: X₁²=0.0015, P>0.9);

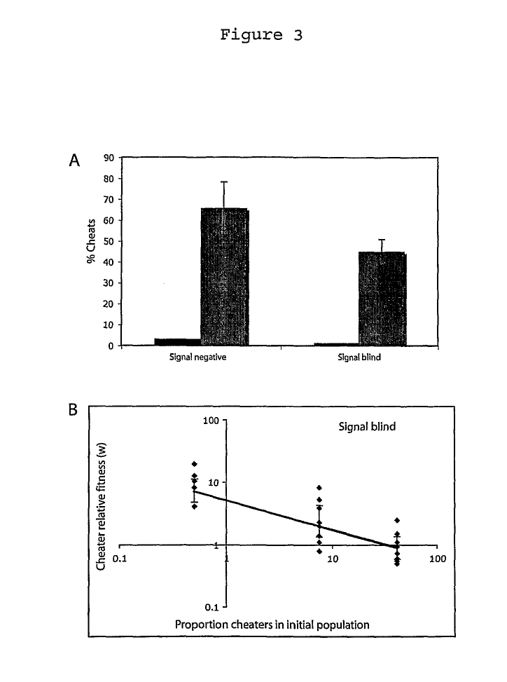

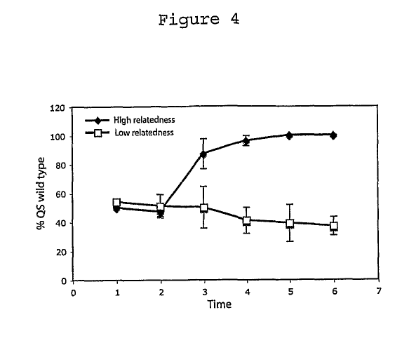

FIG. 3 illustrates that QS is subject to cheating in vivo. QS signal-negative (lasI) and signal-blind (lasR) mutants invade populations of wild-type cooperators during infections of mice. Shown are data for signal-blind mutants in the burn wound model (six mice sacrificed per day, on each of days 1 and 2 after infection), and both signal-blind and signal-negative mutants in the chronic wound model (six mice sacrificed per day for lasR and 3 mice per day for lasI, on each of days 2, 4 and 7 after infection). In all cases, the proportion of cheats significantly increased compared with the initial starting frequency of approximately 1% in the infecting dose (Chronic lasR: P<0.0001, n=18; Chronic lasI: P=0.004, n=9; Burn lasR: P<0.0001, n=18; Wilcoxon ranked sign tests). The same qualitative pattern was also found when the frequency of cheats in the infection dose was varied from 0.1-20%, with the infections examined in FIG. 4 (data not shown). Results shown are means±s.e.m., comparing the initial infection with that on the final day measured (back transformed after arcsine square root transformation);

FIG. 4 illustrates mutant fitness is negatively frequency dependent. The relative fitness of signal-blind (lasR) mutants is plotted against the proportion of mutants used to inoculate the infection, for both burn wound (a) and chronic wound (b) mouse models. In both cases, mutant have a higher fitness when they are less common (Burn wound: F_{(1,16)}=11.20, P=0.004; Chronic wound: F_{(1,16)}=72.58, P<0.0001). Relative fitness is the estimated growth rate of mutants relative to that of the wild type (see methods);

FIG. 5 is the nucleic acid sequence of the Pseudomonas aeruginosa autoinducer synthesis gene lasI;

FIG. 6 is the nucleic acid sequence of the Pseudomonas aeruginosa regulator gene lasR;

FIG. 7 is the nucleic acid sequence of the Pseudomonas aeruginosa autoinducer synthesis gene rhlI;

FIG. 8 is the nucleic acid sequence of the Pseudomonas aeruginosa regulator gene rhlR;

FIG. 9 is the nucleic acid sequence of the Pseudomonas aeruginosa pyoverdine synthesis gene pvdF;

FIG. 10 is the nucleic acid sequence of the Pseudomonas aeruginosa pyochelin synthesis gene pchF; and

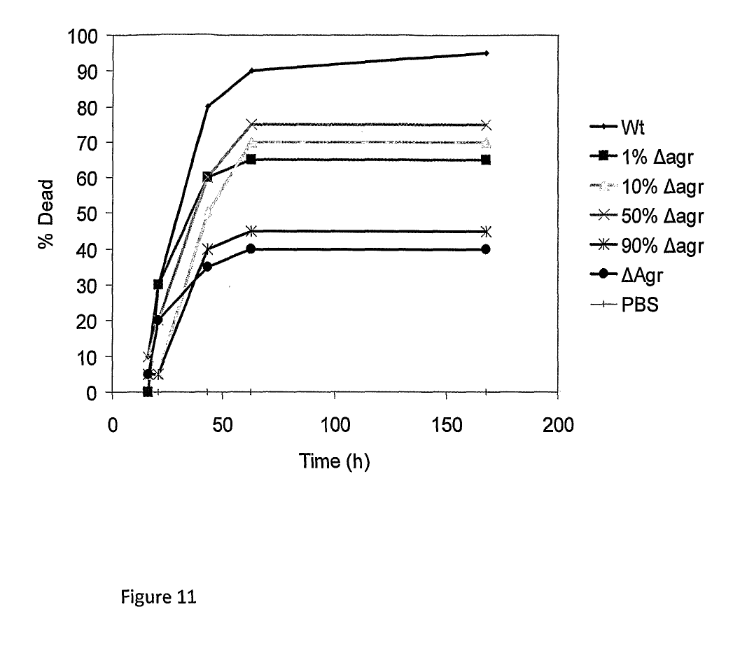

FIG. 11 illustrates the difference between a Quorum sensing Staphylococcus aureus mutant with a wild type Staphylococcus aureus.

### MATERIALS AND METHODS

### Bacterial Growth and Inoculum

P. aeruginosa strains were grown in Luria-Bertani (LB) medium³⁰. Overnight cultures were subcultured in fresh LB broth and grown at 37° C. for 3 h to an optical density of approximately 0.8 at 600 nm. Cultures were serially diluted in sterile phosphate buffered saline (PBS). To generate lasI, lasR, rhlI and rhlR mutants in P. aeruginosa PA14, pSB219.8A (pRIC380 carrying lasI::Gm), pSB219.9A (pRIC380 carrying lasR::Gm), pSB224.12B (pRIC380 carrying rhlI::Tc) and pSB224.10A (pRIC380 carrying rhlR::Tc) were conjugated into PA14 resulting in PA14::/lasI, PA14::/lasR, PA14::rhlI and PA14::rhlR respectively. Mutations were confirmed by PCR analysis (data not shown). To distinguish between wild type and mutant after in vivo competition assays, Mini-CTXlux was transformed into PA14 wild type. This provided a background level of bioluminescence which could be detected under a light camera and so the wild type was bioluminescent whereas the QS mutants were not.

### Acute and Chronic Wound Models

Female Swiss Webster (SW) mice were obtained from Charles River Laboratories (Wilmington, Mass., USA). Mice used in experiments were 6-8 weeks old and weighed 20-25 g. Mice were anesthetized by intraperitoneal injection of Nembutal at 100 mg/kg (5% sodium pentobarbital; Abbott Laboratories, North Chicago, Ill.), and their backs were shaved. The acute 3^{rd} degree burn wound was induced as previously described^{31, 32}. Chronic wounds were induced by the surgical removal of a 1.5×1.5 cm full-thickness patch of skin from the shaved back. The wounds were covered with transparent, semipermeable polyurethane dressing which allowed for daily inspection of the wound, wound size determination, topical application of bacteria onto the wound, and protection from other contaminating bacteria. 10⁴ CFU P. aeruginosa were applied under the dressing, on top of the wound. Mice were treated humanely and in accordance with the protocol approved by the Animal Care and Use Committee at Texas Tech University Health Sciences Center (Lubbock, Tex.).

### Quantitation of Bacteria Within the Skin and Livers

At indicated times mice were euthanized by intracardial injection of 0.2 ml of Sleepaway (sodium pentobarbital-7.8% isopropyl alcohol euthanasia solution; Fort Dodge Laboratories, Inc., Fort Dodge, Iowa). Skin and liver sections from wounded mice were extracted, weighed, placed in 2 ml PBS, and homogenized. Homogenates were serially diluted and plated on LB agar plates to determine the number of bacterial CFU, which was calculated per gram of tissue.

### Statistical Analyses

Unless stated otherwise, we carried out all analyses by model simplification to the minimum adequate model, using generalized linear modeling techniques implemented in GLMStat 6.0 (Kagi Shareware, Ken Beath, Australia). The mouse survival data was analysed by parametric survival analysis in S-Plus 8.0 (Insightful Corp, Seattle, Wash., US), assuming a Weibull distribution (although qualitatively identical results were obtained assuming an exponential distribution). We calculated the relative fitness of mutants (w), by comparing the frequency of mutants at the beginning and end of the experiment. Specifically, w is given by x₂(1−x₁)/x₁(1−x₂), where x₁ is the initial proportion of mutants in the population, and x₂ is their final proportion²⁰. For example, w=2 would correspond to the mutant growing twice as fast as the wild type cooperator.

### Biofilms in Flow Cells and Confocal Laser Scanning Microscopy

The flow cell apparatus comprises two large inverted media bottles connected in series with silicone tubing to the flow cells, an 8 channel peristaltic pump and the effluent waste container. The media bottle is inverted and a negative pressure created in the airspace to reduce bubble formation. Two media bottles feeds three flow cells (3.3 ml/hr/channel) filling 8 flow chambers in total. The peristaltic pump pulls from downstream of the flow chambers to reduce bubble formation and the flow chambers hangs with the inlet at the bottom, to clear bubbles from the flow chambers.

The flow cells are made from cut Perspex and contain 3 parallel flow chambers which are newly covered with a glass coverslip before each experiment. This coverslip forms the substratum of the biofilm and can be easily placed on the microscope stage. Once assembled the flow system is sterilised with 1 L 0.5% (v/v) sodium hypochlorite bleach and rinsed with 2 L sterile dH2O. Following this, the media bottles are attached in a sterile manner and the flow started. The medium is left to flow through the system and saturate the tubing for 24 h before inoculation. Flow cells are inoculated with 250 μl washed culture at OD600 0.1, the flow cells are inverted and incubated for 1 h to allow for initial attachment. Then the pump is restarted.

Biofilms are imaged using a Zeiss LSM 510 Axiovert 100M UV META Kombi microscope (Carl Zeiss, Germany). Imaging is done at 5 mm from the inlet of the chamber. 5 pseudo-replicate image stacks are collected per chamber moving a set amount (5 turns of the remote, ˜500 μm) from the centre and around the edges of a square. Lasers and filters can be combined to excite and detect emissions from green and red fluorescent proteins expressed by different strains.

### Statistics

Biofilm structure is quantified from the confocal stacks using the image analysis software package COMSTAT41. The program was written as a script in MATLAB 5.1 (The MathsWorks Inc., Natick, Mass.), equipped with the Image Processing Toolbox and was originally designed to analyse images from the Leica TCS4D confocal microscope.

### EXAMPLE 1

We examined the virulence of Pseudomonas aeruginosa in the mouse burn model, using a number of QS mutants. P. aeruginosa is an opportunistic pathogen, capable of causing disease in plants and animals, including humans¹⁷. P. aeruginosa pathogenesis in human burn wounds has been extensively examined using the thermally-injured mouse (burn) model, which closely resembles human burn wound sequela. In this acute wound model, a 3^{rd} degree scald burn is produced and a low infecting dose (10² colony forming units (CFU)) of P. aeruginosa causes up to 100% mortality within 48 hours¹⁸. P. aeruginosa regulates production of a number of virulence factors via a complicated hierarchical QS cascade¹⁷. We mutated four key QS genes in PA14, a human clinical isolate of P. aeruginosa that is also capable of causing disease in mice, plants, nematodes and insects¹⁹. We constructed two signal-negative strains that do not produce their cognate autoinducer molecules N-(3-oxododecanoyl)-L-homoserine lactone (3-oxo-C12-HSL; PA14ΔlasI) and N-butanoylhomoserine lactone (C4-HSL; PA14ΔrhlI) and two signal-blind strains that do not respond to autoinducer molecules (PA14ΔlasR and PA14ΔrhlR). We infected mice with either one of these mutants or the wild type from which they originated. Consistent with previous results, we found that the virulence of the mutants, as measured by the rate of host mortality, was significantly lower for the QS mutants (FIG. 1). Overall, these results confirm the group benefit to the infection success provided by QS, and that this benefit is realised because it allows bacteria to better spread within the host¹⁸, and hence lead to more virulent infections.

### EXAMPLE 2

We then tested whether the presence of mutants reduced the virulence in mixed infections that were initiated with a mutant and the wild type. We initiated infections with either 100% PA14 wild type, 100% QS (las) mutant or a 50:50 mixture of the two, repeating this experiment with both the lasR (signal-blind) and lasI (signal-negative) mutants. We focused from here onwards on the las QS mutants, because the las QS pathway controls the rhl system hierarchically (a mutation in las QS results in a general abolition of QS in P. aeruginosa)¹⁷. Furthermore, las mutants are the most commonly detected QS mutant in natural infections of humane¹⁰⁻¹². In both cases the virulence of the 50:50 mix was significantly lower than that of the 100% wild type, and not significantly different from the 100% mutant infections (FIG. 2). The effect on virulence was large, with the addition of mutants to the wild type infections approximately halving the mortality rate over the course of our experiments.

### EXAMPLE 3

We then tested whether the reduced virulence in mixed infections could be explained by social interactions between the wild type and the mutants. Specifically, we tested whether the reduced virulence was due to the mutants exploiting the cooperative behaviour of the wild type, to their benefit, but at the cost of the overall infection. If this is the case, then we can make two predictions. First, in mixed infections, containing both mutants and the wild type, the mutants should increase in frequency because they are able to benefit from (exploit) the QS behaviour of others, whilst avoiding the cost of either signalling or responding to signal. Second, mutants will have a higher fitness when they are more rare (negative frequency dependence), because they will be better able to exploit those that do QS²⁰.

We found support for both of these predictions, in both the acute burn and the chronic wound animal models. We carried out experiments in both models, because, while mortality is high and rapid in the acute model, the chronic wound model allows us to follow infections over a longer time-span. In addition, the models are representative of different types of human P. aeruginosa infection such as burn versus chronic diabetic wounds²¹. We first tested whether QS mutants increase in frequency in mixed infections. We initiated infections of the wild type with approximately 1% of either the signal-blind (lasR−; both models) or signal-negative (lasI−; just chronic model) mutants. In the chronic wound model, over a period of 7 days, the signal-blind mutant increased in frequency from 1.3% to 32.4%, and the signal-negative mutant increased in frequency from 1.0% to 13.4% (FIG. 3). Consequently, in mixed infections, both the signal-blind and the signal-negative mutants had a significantly higher fitness than the parental wild type—the relative fitness of the two mutants were 47-fold and 16-fold that of the wild type, respectively. In the acute burn model, over a period of two days, the signal-blind mutant increased in frequency from 1.4% to 14.3% (FIG. 3), with the relative fitness of the mutant being 13 times that of the wild type.

### EXAMPLE 4

We then tested whether the success of the mutants showed frequency dependence, with mutants having a lower relative fitness when they are more common. Social evolution theory predicts that if the mutants and wild type are cheats and cooperators respectively, then the relative fitness of the mutants will be greatest when they are more rare, because then they will be better able to exploit the cooperators^{20, 22}. This is because (a) a higher proportion of cooperators will lead to greater population growth, allowing more time for cheats to exploit cooperators, and (b) population structuring will reduce the extent to which the cheats and cooperators interact, which penalises cheats more at higher frequencies²⁰. Both of these factors are likely to be important within hosts. We initiated infections of the wild type, in both the acute burn and chronic wound model, with 0.1%, 1% or 20% of the signal-blind (lasR−) mutant. In both cases, as predicted, we found that the relative success of the signal-blind cheat was lower when it was more common (FIG. 4). Although the mutant increased in frequency from all starting frequencies, its relative fitness was greater when at lower starting frequencies (see methods).

This experiment also suggests that the spread of cheats to new areas of the host can be facilitated by their exploitation of the cooperative behaviour of the wild type. It has previously been shown that QS mutants are inhibited in their ability to cause systemic infections and thus reach the liver¹⁸. We examined the spread of bacteria to the liver in 18 burned mice, where we had initiated mixed infections, with both the wild type and the signal blind (lasR) mutant. Consistent with previous work, we found that, after 24 hours, P. aeruginosa was present in the livers of 39% of the mice (7/18), and that these liver infections were entirely wild type bacteria (0% signal-blind (lasR) mutant). By 48 hours, 100% of mice (18/18) had P. aeruginosa in their livers. However, in these mice, all of the livers had also been invaded by the signal-blind (lasR) mutant, which had risen from 0% to an average of 11.7% (95% C.I: 8.1-15.7%) of the bacteria in the liver infections. Consequently, this shows that once the liver is colonised by wild type bacteria, the mutants are then able to invade and significantly increase in frequency (F_{(1,23)}=51.47, P<0.0001).

Overall, our results support the hypothesis that QS mutants spread in natural infections, because they are cheats, which are able to exploit the cooperative signalling and exoproduct production of the wild type. QS mutants, especially signal-blind (lasR) mutants, are commonly found in clinical settings, such as the lungs of humans with cystic fibrosis¹⁰. The alternative explanation for the spread of such QS mutants is that they are better adapted to the host environment²³—i.e. a direct rather than social benefit (although both are possible). However, if this was the case, then we would expect: (a) infections of mutants to spread better and be more virulent than infections of the wild type, and (b) the invasion of mutants in mixed infections to lead to increased growth and virulence. In contrast to these predictions, the opposite patterns occur, with mutants leading to a reduction in both the spread¹⁸ and virulence (FIGS. 1 & 2) of infections. In addition, if the spread of mutants was due to adaptation to the host environment, then we would not predict the observed pattern of frequency dependence (FIG. 4). More generally, our results confirm that QS can be a social trait in a natural environment, and that signalling between cells is not just an artefact of laboratory culture methods, such as artificially high densities^{8, 9}.

Our results support the idea that parasite virulence in bacteria can be driven by cooperative interactions, which may explain the lack of a consistent pattern in the influence of strain diversity (relatedness) on parasite virulence^{14, 24-27}. Specifically, a negative correlation between virulence and strain diversity would be predicted if host exploitation is limited by the extent of cooperation¹⁴, and a positive correlation would be predicted if prudent exploitation of host resources is more important^{15, 16, 28}. This contrasts with related areas of social evolution theory that have been applied to parasites, such as sex ratio adjustment in response to competition between related males, where the biological details do not have a strong influence on the predictions of theory²⁹. Finally, our results raise the potential for social interactions to be exploited in medical interventions. Cheats that do not perform cooperative behaviours could be introduced into hosts, to outcompete wild type cooperators. As well as the direct benefit of reducing virulence, this could drive down the bacterial population size, which may benefit other intervention strategies such as treatment with antibiotics.

### EXAMPLE 5

In order to study the quorum sensing signal molecule production of clinical P. aeruginosa isolates, 43 strains were isolated from a cohort of 36 individual patient sputum samples. 16 of these strains came from samples provided by paediatric patients. Each sputum sample was plated onto selective agar.

All strains were confirmed to be rapidly oxidase positive Gram-negative rods. Twelve strains (28%) grew as mucoid colonies on Pseudomonas isolation agar (PIA) after incubation at 37° C. for 24 to 48 h. The remaining 31 (72%) were non mucoid. The proportion of mucoid strains was similar for the subset of strains obtained from adult patients and for those obtained from paediatric patients at 26% and 31% respectively. Of the 6 cases in which a pair of isolates was recovered from a single sputum sample, 5 of these consisted of a non-mucoid strain co-existing with a mucoid isolate. The majority of strains (31; 72%) produced the green pigment pyocynin. Of those remaining, 5 (12%) produced a pink pigment thought to be pyorubin and 7 (16%) were colourless, suggesting no pigment production.

The cohort of 43 clinical isolates was investigated for their ability to produce the quorum sensing signal molecules 3O-C12-HSL, C4-HSL, HHQ and PQS when grown as broth cultures; Table 1. TLC analysis was undertaken in conjunction with specific signal molecule sensor bacteria. TLC plates were selected to allow the optimal separation of each signal molecule in an appropriate solvent system and then overlaid with a lawn of reporter bacteria. C4-HSL was produced by all isolates bar one obtained from an adult patient. In total, 22 (51%) isolates were deficient in 3O-C12-HSL production. A deficiency in 3O-C12-HSL suggests that a defective las QS system exists in these strains. These could therefore be considered to be naturally occurring cheats that are similar to the genetically engineered cheats described by Diggle et al. (Diggle et al. 2007. Nature. 450: 411-414). Preliminary sequencing data suggests that at least some of these isolates contain point mutations in the lasR gene and so are therefore defective in the ‘response’ to QS and are thus like the signal blind strains described in Diggle et al. (2007). Examining the distribution of these signal molecule-deficient isolates, it was apparent that 37% (10/27) of adult isolates lacked 3O-C12-HSL compared to 75% (12/16) of isolates obtained from paediatric patients. HHQ was detected for all isolates and 95% (41) of isolates produced PQS.

### EXAMPLE 6

We have successfully set up a model to assess Staphylococcus aureus virulence using Galleria mellonella (Waxmoth) larvae, showing reproducible killing over 7 days at 37° C. These represent normal physiological conditions for the bacteria and are thus of relevance to human infections. We have successfully used this model to study the virulence of a S. aureus wild type (wt) and a quorum sensing (QS) mutant strain, Δagr. It is known in the literature that the agr system regulates a number of toxins important for virulence. As shown in FIG. 11, a strain lacking the agr genes is attenuated in virulence over the course of the assay, whereas the QS producer strain of S. aureus causes more rapid death of the insects. This strain also kills a larger percentage of the insects over the course of the assay in comparison to the QS non-producer. Using different proportions of QS producing and non QS producing strains in the inoculum, we have shown that the inclusion of a small proportion of cheat in the population reduces the pathogenicity of the community as a whole.

TABLE 1
Summary of clinical isolate signal molecule status:

| Strain | C4 | C12 | HHQ | PQS |
|---|---|---|---|---|
| A001-200804 | y | y | y | y |
| A002-051104 | y | y | y | y |
| A003-280504 | y | y low | y | y |
| A004-130804 | y | y | y | y |
| A005-100904 | y | y low | y | y |
| A007-110604 | y | y | y | y |
| A009-110604 | y | y low | y | y |
| A012-180604A | y | n | y | y |
| A012-180604B | n | n | y | y |
| A014-291004 | y | y | y | y |
| A017-081004 | y | n | y | y |
| A018-151004 | y | y | y | y |
| A019-040205 | y | n | y | y |
| A021-101204A | y | y | y | y |
| A021-101204B | y | n | y | y |
| A023-200804 | y | y | y | y |
| A024-270804 | y | n | y | y |
| A025-221004 | y | y | y | y |
| A026-130804 | y | n | y | y |
| A029-110305 | y | n | y | y |
| A031-030904 | y | n | y | y |
| A032-200804 | y | n | y | y |
| A033-200804 | y | y | y | y |
| A035-051104A | y | y | y | y |
| A035-051104B | y | y | y | y |
| A037-230205A | y | y | y | y |
| A037-230205B | y | y | y | y |
| P003-170804 | y low | n | y | n |
| P004-010205 | y | y low | y | y |
| P006-170804 | y low | n | y | n |
| P007-280904 | y | n | y | y |
| P009-280904 | y | n | y | y |
| P010-191004 | y | y | y | y |
| P010-211204 | y | n | y | y |
| P013-101204A | y | n | y | y |
| P013-101204B | y | n | y | y |
| P015-170804A | y | n | y | y |
| P015-170804B | y | n | y | y |
| P016-280904 | y | n | y | y |
| P018-161104 | y | n | y | y |
| P020-191004 | y | y | y | y |
| P021-211204 | y | n | y | y |
| P024-070904 | y | y | y | y |

### REFERENCES

1. Keller, L. & Surette, M. G. Communication in bacteria: an ecological and evolutionary perspective. Nat. Rev. Microbiol. 4, 249-58 (2006).
2. Diggle, S. P., Gardner, A., West, S. A. & Griffin, A. S. Evolutionary theory of bacterial quorum sensing: when is a signal not a signal? Philos. Trans. R. Soc. Lond. B. Biol. Sci. 362, 1241-9 (2007).
3. Henke, J. M. & Bassler, B. L. Bacterial social engagements. Trends. Cell. Biol. 14, 648-56 (2004).
4. Williams, P., Winzer, K., Chan, W. C. & Camara, M. Look who's talking: communication and quorum sensing in the bacterial world. Philos. Trans. R. Soc. Lond. B. Biol. Sci. 362, 1119-34 (2007).
5. Brown, S. P. & Johnstone, R. A. Cooperation in the dark: signalling and collective action in quorum-sensing bacteria. Proc. Biol. Sci. 268, 961-5 (2001).
6. Diggle, S. P., Griffin, A. S., Campbell, G. S. & West, S. A. Cooperation and conflict in quorum-sensing bacterial populations. Nature 450, 411-4 (2007).
7. Sandoz, K. M., Mitzimberg, S. M. & Schuster, M. Social cheating in Pseudomonas aeruginosa quorum sensing. Proc. Natl. Acad. Sci. USA 104, 15876-81 (2007).
8. Redfield, R. J. Is quorum sensing a side effect of diffusion sensing? Trends Microbiol. 10, 365-70 (2002).
9. Hense, B. A. et al. Does efficiency sensing unify diffusion and quorum sensing? Nat. Rev. Microbiol. 5, 230-9 (2007).
10. Smith, E. E. et al. Genetic adaptation by Pseudomonas aeruginosa to the airways of cystic fibrosis patients. Proc. Natl. Acad. Sci. USA 103, 8487-92 (2006).
11. Schaber, J. A. et al. Analysis of quorum sensing-deficient clinical isolates of Pseudomonas aeruginosa. J. Med. Microbiol. 53, 841-53 (2004).
12. Denervaud, V. et al. Characterization of cell-to-cell signaling-deficient Pseudomonas aeruginosa strains colonizing intubated patients. J. Clin. Microbiol. 42, 554-62 (2004).
13. Smith, R. S. & Iglewski, B. H. P. aeruginosa quorum-sensing systems and virulence. Curr. Opin. Microbiol. 6, 56-60 (2003).
14. Brown, S. P., Hochberg, M. E. & Grenfell, B. T. Does multiple infection select for raised virulence? Trends Microbiol. 10, 401-5 (2002).
15. Frank, S. A. Models of parasite virulence. Q. Rev. Biol. 71, 37-78 (1996).
16. Herre, E. A. Population Structure and the Evolution of Virulence in Nematode Parasites of Fig Wasps. Science 259, 1442-1445 (1993).
17. Venturi, V. Regulation of quorum sensing in Pseudomonas. FEMS Microbiol. Rev. 30, 274-91 (2006).
18. Rumbaugh, K. P., Griswold, J. A., Iglewski, B. H. & Hamood, A. N. Contribution of quorum sensing to the virulence of Pseudomonas aeruginosa in burn wound infections. Infect. Immun. 67, 5854-62 (1999).
19. Rahme, L. G. et al. Common virulence factors for bacterial pathogenicity in plants and animals [see comments]. Science 268, 1899-902 (1995).
20. Ross-Gillespie, A., Gardner, A., West, S. A. & Griffin, A. S. Frequency dependence and cooperation: theory and a test with bacteria. Am. Nat. 170, 331-42 (2007).
21. Bodey, G. P., Bolivar, R., Feinstein, V. & Jadeja, L. Infections caused by Pseudomonas aeruginosa. Rev. Infect. Dis. 5, 279-313 (1983).
22. MacLean, R. C. & Gudelj, I. Resource competition and social conflict in experimental populations of yeast. Nature 441, 498-501 (2006).
23. D'Argenio, D. A. et al. Growth phenotypes of Pseudomonas aeruginosa lasR mutants adapted to the airways of cystic fibrosis patients. Mol. Microbiol. 64, 512-33 (2007).
24. Read, A. F. & Taylor, L. H. The ecology of genetically diverse infections. Science 292, 1099-102 (2001).
25. Foster, K. R. Biomedicine. Hamiltonian medicine: why the social lives of pathogens matter. Science 308, 1269-70 (2005).
26. Buckling, A. & Brockhurst, M. A. Kin selection and the evolution of virulence. Heredity 100, 484-8 (2008).
27. Frank, S. A. & Schmid-Hempel, P. Mechanisms of pathogenesis and the evolution of parasite virulence. J. Evol. Biol. 21, 396-404 (2008).
28. de Roode, J. C. et al. Virulence and competitive ability in genetically diverse malaria infections. Proc. Natl. Acad. Sci. USA 102, 7624-8 (2005).
29. Nee, S., West, S. A. & Read, A. F. Inbreeding and parasite sex ratios. Proc. Biol. Sci. 269, 755-60 (2002).
30. Ausubel, F., R. Brent, R. Kingston, D. Moor, J. Seidman, J. Smith, and K. Stauhle. Current Protocols in molecular biology (Wiley Intersciences, New York, N.Y., 1988).
31. Haynes, A., 3rd et al. Syndecan 1 Shedding Contributes to Pseudomonas aeruginosa Sepsis. Infect Immun 73, 7914-21 (2005).
32. Schaber, J. A. et al. Pseudomonas aeruginosa Forms Biofilms in Acute Infection Independent of Cell-to-Cell Signaling. Infect Immun 75, 3715-21 (2007).

1.-48. (canceled)

49. A method of inhibiting infection by a bacteria in a subject, said method comprising, administering to said subject a cell of said bacteria having a genome which is modified by addition, deletion or substitution of at least one nucleotide base in at least one site in said genome such that said bacteria is defective in the expression of at least one gene, thereby inhibiting said bacterial infection.

50. The method according to claim 49 wherein said subject is a member selected from a human, a non-human animal and a plant.

51. The method according to claim 49 wherein said bacteria is a member selected from Gram positive and Gram negative bacteria.

52. The method according to claim 49 wherein said bacteria is a member selected from: Enterococcus spp; Mycobacterium spp; Streptococcus group B spp; Streptoccocus spp; Helicobacter spp; Neisseria spp; Streptococcus group A spp; Borrelia spp; Coccidiodes spp; Histoplasma spp; Klebsiella spp; Proteus spp; Shigella spp; Escherichia spp; Haemophilus spp; Chalmydia spp; Francisella spp; Pseudomonas spp; Bacillus spp; Clostridium spp; Yersinia spp; Burkholderia spp; Agrobacterium; Pseudomonas spp; and Staphylococcus spp.

53. The method according to claim 52 wherein said bacteria is selected from the group Pseudomonas aeruginosa; P. oryzihabitans; P. syringae, P. tolaasii; P. agarici; P. luteola; Staphylococcus aureus; S. epidermidis, S. hominis; S. haemolyticus; S. warneri; S. capitis; S. saccharolyticus; S. auricularis; S. simulans; S. saprophyticus; S. cohnii; S. xylosus; S. cohnii; S. warneri; S. hyicus; S. caprae; S. gallinarum; S. intermedius; and S. hominis

54. The method according to claim 49, wherein said genome modification is to an operon comprising a gene encoding a polypeptide that mediates quorum sensing.

55. The method according to claim 54 wherein said operon is a member selected from the las operon, and the rhl operon.

56. The method according to claim 55 wherein said gene is a member selected from lasI; rhlI; lasR; rhlR and a combination thereof.

57. The method according to claim 49 wherein said bacterial infection is selected from septicaemia; tuberculosis; bacteria-associated food poisoning; blood infections; peritonitis; endocarditis; sepsis; bacterial meningitis; pneumonia; stomach ulcers; gonorrhoea; strep throat; streptococcal-associated toxic shock; necrotizing fasciitis; impetigo; histoplasmosis; Lyme disease; gastro-enteritis; dysentery; and shigellosis

58. The methods according to claim 49 wherein an antibiotic is administered to said subject with said bacterial cell.

59. A bacterial cell comprising a genome modified by addition, deletion or substitution of at least one nucleotide base in at least one site in the genome wherein said modification causes a defect in expression of a polypeptide which mediates quorum sensing, said gene comprising a nucleic acid sequence which is a member selected from:
a nucleic acid molecule comprising a nucleic acid sequence as represented in FIG. 5;
a nucleic acid molecule comprising a nucleic acid sequence that hybridizes under stringent hybridization conditions to the nucleic acid molecule in i) above and encodes a polypeptide with the activity of las autoinducer;
a nucleic acid molecule comprising a nucleic acid sequence as represented in FIG. 6;
a nucleic acid molecule comprising a nucleic acid sequence that hybridizes under stringent hybridization conditions to the nucleic acid molecule in iii) above and encodes a polypeptide with the activity of las regulator.
a nucleic acid molecule comprising a nucleic acid sequence as represented in FIG. 7;
a nucleic acid molecule comprising a nucleic acid sequence that hybridizes under stringent hybridization conditions to the nucleic acid molecule in v) above and encodes a polypeptide with the activity of a rhlI autoinducer;
a nucleic acid molecule comprising a nucleic acid sequence as represented in FIG. 8;
a nucleic acid molecule comprising a nucleic acid sequence that hybridizes under stringent hybridization conditions to the nucleic acid molecule in i) above and encodes a polypeptide with the activity of rhlI regulator;
a nucleic acid molecule comprising a nucleic acid sequence as represented in FIG. 9;
a nucleic acid molecule comprising a nucleic acid sequence that hybridizes under stringent hybridization conditions to the nucleic acid molecule in ix) above and encodes a polypeptide with siderophore activity;
a nucleic acid molecule comprising a nucleic acid sequence as represented in FIG. 10; and
a nucleic acid molecule comprising a nucleic acid sequence that hybridizes under stringent hybridization conditions to the nucleic acid molecule in xi) above and encodes a polypeptide with siderophore activity.

60. A bacterial cell according to claim 59 wherein said modified bacterial cell is additionally modified by transformation with a nucleic acid molecule that encodes an agent which when expressed sensitizes said cell to an agent that inhibits the growth of said modified cell.

61. A method of treating a wound in a subject comprising administering to said subject a bacterial cell the genome of which is modified by addition, deletion or substitution of at least one nucleotide base in at least one site in the genome such that said bacterial cell is defective in the expression of at least one gene, thereby treating said wound.

62. The method according to claim 61 wherein said wound has a pre-existing bacterial infection.

63. The method according to claim 61 wherein said wound is a member selected from a burn, scald and an ulcer.

64. The method according to claim 63 wherein said ulcer is a diabetic ulcer.
